(19) United States
(12) Patent Application Publication
(10) Pub. No.: US 2015/0291924 A1
(43) Pub. Date: Oct. 15, 2015

(54) FERMENTER OF A BIOGAS SYSTEM

(71) Applicant: UTS BIOGASTECHNIK GMBH, Hallbergmoos (DE)

(72) Inventors: Andreas Czwaluk, Vechta (DE); Johann Bierer, Dorfen (DE)

(73) Assignee: UTS BIOGASTECHINK GMBH, Hallbergmoos (DE)

(21) Appl. No.: 14/439,881

(22) PCT Filed: Oct. 30, 2013
(86) PCT No.: PCT/EP2013/072745

§ 371 Date: Apr. 30, 2015

(30) Foreign Application Priority Data
Oct. 30, 2012 (DE) 10 2012 021 206.2

(51) Int. Cl. C12M 1/107; C12M 1/06; C12M 1/34; B01F 15/00

(52) U.S. Cl. CPC C12M 21/04; B01F 15/00694; C12M 27/02; C12M 41/42; B01F 2215/0073

(57) ABSTRACT

Digester of a biogas plant including a digester wall and a digester interior and a digester roof inclined to the horizontal and flexible in design and including an agitator device for stirring a substrate in the digester interior. The agitator device comprises a guiding unit, a support unit, and an adjustable agitating unit accommodated at the guiding unit. The support unit is fastened to the digester wall for supporting the guiding unit. The agitating unit is provided to be height-adjustable and an adjustment device is provided for displacing the agitating unit. The top end of the guiding unit extends up to and beyond a top end of the digester wall and terminates beneath the digester roof within the digester interior.

The present invention relates to a digester of a biogas plant having at least one digester wall and at least one digester interior and a digester roof. An agitator device is disposed in the interior of the digester for agitating the substrate in the digester interior. The agitator device is adjustably disposed on a guide rod which acts as a guiding unit and is in particular disposed perpendicular.

The prior art has disclosed a great variety of biogas plants and digesters. The substrate in the digester is as a rule agitated or stirred to cause the substrate to homogenize and to prevent various components from settling. In operation the orientation of the agitator device requires regular realignment to flush dead spaces and to achieve thorough mixing. The agitator device or agitator may be realigned on a regular basis e.g. once in 10 or 14 days, the interval being dependent on the substrate and other given conditions. Agitators used for agitating tend to be submersible agitators, having 2, 3 or 4 agitator blades. The agitator and in particular the agitator blades are subject to wear and therefore require maintenance on a regular basis. During such maintenance e.g. one or all of the agitator blades may be changed.

To facilitate maintenance and to prevent the filling level in the digester from dropping too low during maintenance, these agitators are as a rule accommodated height-adjustable in the digester. For maintenance the agitator is displaced upwardly so as to provide better access to the agitator or the agitator blades.

To facilitate maintenance and also height adjustment and angular adjustment of the agitator, EP 1 717 305 B1 has disclosed a servicing shaft for a digester tank of a biogas plant penetrating the flexible film roof gastight. The agitator is retained on a vertical supporting rod that extends from the digester bottom up into the servicing shaft. For maintenance purposes the agitator may be displaced upwardly into the servicing shaft so that the agitator is conveniently accessible for maintenance purposes after opening the servicing shaft doors.

Document DE 19 732 198 C1 has disclosed a lifting device for a submersible motor agitator of a digester tank of a biogas plant wherein the digester tank is covered by a concrete roof that has an opening adequately sized for transferring the submersible motor agitator out of the digester tank into a domed tank. There the submersible motor agitator is conveniently accessible for maintenance purposes after opening the dome. The height of the submersible motor agitator is adjusted via a cable spool drive, the cable spool being disposed within the edge region and driven by an electric motor. Thus there is no need for a gastight passage of the rope through the tank wall.

The prior art described operates most satisfactorily. However, the use of concrete roofs rather increases the structural work involved in such a biogas plant. A servicing shaft in a flexible film roof allows much greater ease of servicing an agitator and moreover also facilitates adjusting the agitator. The structural work involved in such a servicing shaft also increases the price though.

More cost-effective digesters have been disclosed; for example document DE 2009 001 118 U1 discloses an adjustment device for submersible motor agitators where a rope for adjusting the height of the submersible motor agitator passes outwardly through the tank wall through a tube constructed as a piston rod. On the outside of the tank wall a manual rope winch is provided which when actuated lowers or raises the submersible motor agitator. Axial displacement of the piston rod further influences the rotational angle of the submersible motor agitator. This prior art is basically also functional. However, sealing the rope passage through the tank wall requires regular lubrication of the piston rod which acts as the guide tube to ensure gas tightness. The longitudinal movement of the rope through the piston rod which serves as the guide tube makes it difficult in this design to ensure the tightness required. Other designs have disclosed a guide rope passing through a water cup or the like to ensure gas tightness in the way of a siphon. Such a water cup requires inspection on a regular basis since for one, the water cup may dry up in particular in summer, while very cold temperatures may cause the water cup to freeze.

Another drawback of the prior art according to DE 2009 001 118 U1 and similar digester structures is that although the agitator can be pulled upwardly for maintenance, it will still be located somewhat beneath the top of the tank wall even in the topmost position. This is why the substrate level may have to be lowered prior to maintenance, and maintenance itself is considerably more difficult than when using a servicing shaft according to EP 1 171 305 B1.

It is therefore the object of the present invention to provide a digester tank that can be manufactured at low cost and which allows ease of servicing the agitating unit.

This object is solved by a digester having the features of claim 1 and by an agitator device having the features of claim 26. Preferred specific embodiments are the subject of the subclaims. Further advantages can be taken from the general description and the description of the exemplary embodiments.

A digester according to the invention is in particular provided for a biogas plant and comprises at least one digester wall and at least one digester interior. The digester comprises at least a digester roof that is at least partially laterally inclined to the horizontal and is in particular flexible in configuration. At least one agitator device is provided for stirring a substrate in the digester interior. The agitator device comprises at least one guiding unit, at least one support unit, and at least one adjustable agitating unit accommodated on the guiding unit. The support unit for supporting the guiding unit is fastened to the digester wall. The agitating unit is provided to be height-adjustable. At least one adjustment device is provided for adjusting the agitating unit. The top end of the guiding unit extends up to beneath a top end of the digester wall and terminates beneath the digester roof within the digester interior.

The digester according to the invention has many advantages. A considerable advantage of the digester according to the invention consists in allowing ease of operation and ease of maintenance. The guiding unit may for example comprise a guide rod or a supporting mast and also a gear transmission and/or a strut or holder, the guiding unit extending up to above the top edge of the digester wall. Since the digester roof rises toward the center at an appropriate angle, the guiding unit can extend beyond the top edge of the digester wall without penetrating the digester roof. These structural measures allow displacement of the agitating unit further upwardly than do other constructions. Other prior art constructions having no servicing shaft show guiding units terminating beneath the digester wall top edge. This means that, at least during maintenance, the digester cannot be filled up as far as is otherwise possible. This means that prior to maintenance, substrate needs to be removed from the digester at considerable efforts. Otherwise the digester capacity is not utilized as fully as possible. Therefore the invention allows a higher degree of utilization and lower expenditure of operation.

Particularly preferably at least part of the agitating unit can be displaced upwardly beyond the top end of the digester wall. In particular at least one agitator blade is provided displaceable at least in part beyond the top end of the digester wall. Particularly preferably at least part of the agitating unit housing is provided displaceable upwardly beyond the top end of the digester wall. This allows particular ease of servicing the agitating unit.

Particularly preferably the agitating unit can be displaced upwardly far enough for the rotation axis of the agitating unit to extend beyond the top end of the digester wall. In particularly preferred configurations the agitating unit is substantially and in particular entirely displaceable beyond the top end of the digester wall. These configurations allow particular ease of accessibility to the agitating unit.

Preferably the support unit comprises at least one supporting rod extending upwardly from the digester wall at an incline. In particular at least two support units are provided each extending upwardly from the digester wall at an incline. Each of the support units comprises in particular at least one supporting rod. Viewed in a top view it is preferred for the two support units to extend in a V-shape toward the tank wall. This allows stability of construction while at the same time enabling the agitating unit to be displaced upwardly between the two support units.

In preferred configurations the adjustment device comprises at least one control unit disposed outside the digester interior and at least one adjusting unit disposed inside the digester interior. The control unit is in particular coupled with the adjusting unit via a gastight passage device. The passage device may be configured as a transmission unit. In simple cases the passage device is a rotatable shaft accommodated gastight. Rotational motions can be kept gastight more easily than can a linear rope passage.

Preferably the guiding unit comprises at least one supporting mast and at least one transmission device. The transmission device can in particular be another or a separate add-on part fastened or attached on the top of the supporting mast.

Preferably the transmission unit serves to adjust in height the agitator device along the supporting mast and/or to pivot the agitator on the supporting mast. Other means may be employed which are suitable to effect displacement or pivoting but are not transmissions in their proper sense.

In preferred configurations the adjusting unit is in functional connection with the transmission unit. Thus, the agitator unit can be easily displaced by means of the control unit outside the digester.

Preferably the adjusting unit is mechanical, hydraulic, and/or pneumatic in configuration. In this case the control unit can displace or pivot the agitator unit e.g. by means of rods or a rod mechanism or else by means of hydraulic or pneumatic pressure.

Particularly preferably the transmission device is configured as a hydraulic transmission and the adjusting unit is provided to be at least one hydraulic hose. Then the hydraulic hose preferably connects the hydraulic transmission with the control unit, the control unit in particular comprising at least one hydraulic pump. This hydraulic pump may e.g. be installed stationary and may be configured as a manual pump or a motorized pump. Or else, the control unit may preferably be provided with an interface where a hydraulic pump can be connected. Then for example this interface may be provided with hydraulic hoses connected thereto so they can be guided to any desired spot.

Then a mobile hydraulic pump may be provided e.g. on the floor adjacent to the digester. This is in particular also suitable for maintenance purposes. It is also preferred for the hydraulic hoses connected to the interface to be guided into a service room where they are connected to a motorized hydraulic pump.

In other preferred configurations the adjustment device is mechanical with the adjusting units in particular configured as rods. Then the control unit preferably comprises a crank device which may be configured as a manual crank or else as an interface for connecting a motorized crank device.

In all the configurations it is particularly preferred for the gastight passage device to be arranged beneath the digester roof and in particular in the digester wall. The gastight passage device allows simple and reliable operation of the digester. The film roof may be designed independently of the provided maintenance function. It is not required to provide a gastight passage of components through the film roof. A gastight passage device may be incorporated into the tank wall or the digester wall in a simple way.

The control unit is particularly preferably positioned beneath the top end of the guiding unit. The control unit is in particular also positioned beneath the top end of the digester wall. In this configuration the control unit is provided beneath the top end of the guiding unit.

The adjusting unit may comprise at least one deflecting device in particular for bridging such a large difference. The deflecting device may comprise, or be configured as, at least one cardan joint device and/or at least one angular drive device and/or at least one cable respectively flexible shaft. Such a deflecting device in the shape of e.g. a cardan joint device or the like allows a reliable and simple transmission of rotational movements performed at an angle to one another. This allows to couple a coupling unit inclined to the horizontal and extending upwardly from the gastight passage device via the deflecting device. This allows to transmit manual operations in the control unit from the region of the tank wall upwardly.

The coupling unit is positioned in particular between the deflecting device in the shape e.g. of a cardan joint device and the passage device.

In all the configurations the agitating unit is provided in particular pivotable. The rotational angle is adjustable by way of an angle adjustment device. The adjustment device for the angle may be configured in analogy so that other than the height adjustment device, a second angle adjustment device is provided.

In all the configurations a distance of the guiding unit from the inside surface of the digester wall is preferably dimensioned such that the agitating unit is pivotable toward the digester wall. The distance of the guiding unit from the inside surface of the digester wall is larger than is the corresponding extension of the agitating unit. Particularly preferably the agitating unit is designed to pivot approximately 360°. The rotational angle may be smaller or larger than 360°.

Preferably the outside surface of the digester wall is provided with at least one platform where it is in particular attached to the outside surface. This platform may be used for example for servicing the agitating unit. An operator may stand on the platform and from there service the agitating unit. It is preferred for at least one platform to be disposed in particular removable at least at one support unit.

In all the configurations it is preferred for at least one maintenance opening, which can be locked gastight, to be provided in the digester roof. Such a lockable maintenance opening is in particular provided above the guiding unit. This configuration allows particular ease and speed of servicing the agitator since the agitator or the agitating unit is displaced upwardly and thereafter the lockable maintenance opening is opened so that the agitating unit is accessible from outside.

The servicing opening preferably comprises a servicing frame or is formed in a servicing frame. This allows to make a servicing opening in a film roof in a simple way.

Advantageous configurations provide for at least one safety bar device beneath the digester roof. The safety bar device is particularly preferably disposed above the guiding device at least in sections.

The safety bar device may in particular be designed as a safety bar that is preferably fastened to the digester wall by both its ends and that may be connected with the guiding unit. In other advantageous configurations the safety bar device may remain unconnected with the digester wall, being only accommodated in the guiding device.

The safety bar device covers in particular the top portion of the guiding unit so that the film roof is protected from any damage through the guiding device in case that the roof collapses due to pressure drop.

In other preferred configurations the safety bar may be provided in the top region of the guiding unit to which it may be attached in particular exclusively. This also achieves adequate protection of the film roof.

The servicing frame is preferably accommodated on the safety bar device. Thus the servicing frame may resist tensions in the film roof and a particularly stable servicing opening is provided.

In all the configurations described above the adjustment device can preferably be operated manually and/or motorized. Thus, depending on the available budget, less expensive systems may be composed and it is moreover possible to adjust the agitator device even in case of power failure or a defective motor.

The agitator device according to the invention is in particular provided to be employed in a digester for a biogas plant for stirring a substrate present in the digester interior. The agitator device comprises at least one guiding unit and at least one support unit. The guiding unit accommodates an adjustable agitating unit and the support unit is, or can be, fastened to a digester wall for supporting the guiding unit. At least one adjustment device is provided for adjusting the agitating unit. The adjustment device comprises at least one adjusting unit connected with at least one control unit via at least one gastight passage device.

The agitator device according to the invention also has many advantages since it allows simple and reliable operation requiring little efforts.

Preferably an adjusting unit for adjusting a height of the agitating unit is provided. In particular an adjusting unit for adjusting an angle of the agitating unit is provided. Particularly preferably the agitating unit is disposed height-adjustable and pivotable.

In useful specific embodiments at least one adjusting unit is mechanical, hydraulic, or pneumatic in design.

The passage device may in particular be configured as, or comprise, an articulated passage.

In all the configurations at least one adjustment device preferably comprises at least one deflecting device and/or a cardan joint device.

Further advantages and applications of the present invention can be taken from the exemplary embodiment which will be described below with reference to the enclosed figures.

The figures show in:

FIG. 1 a schematic side view of a digester according to the invention;

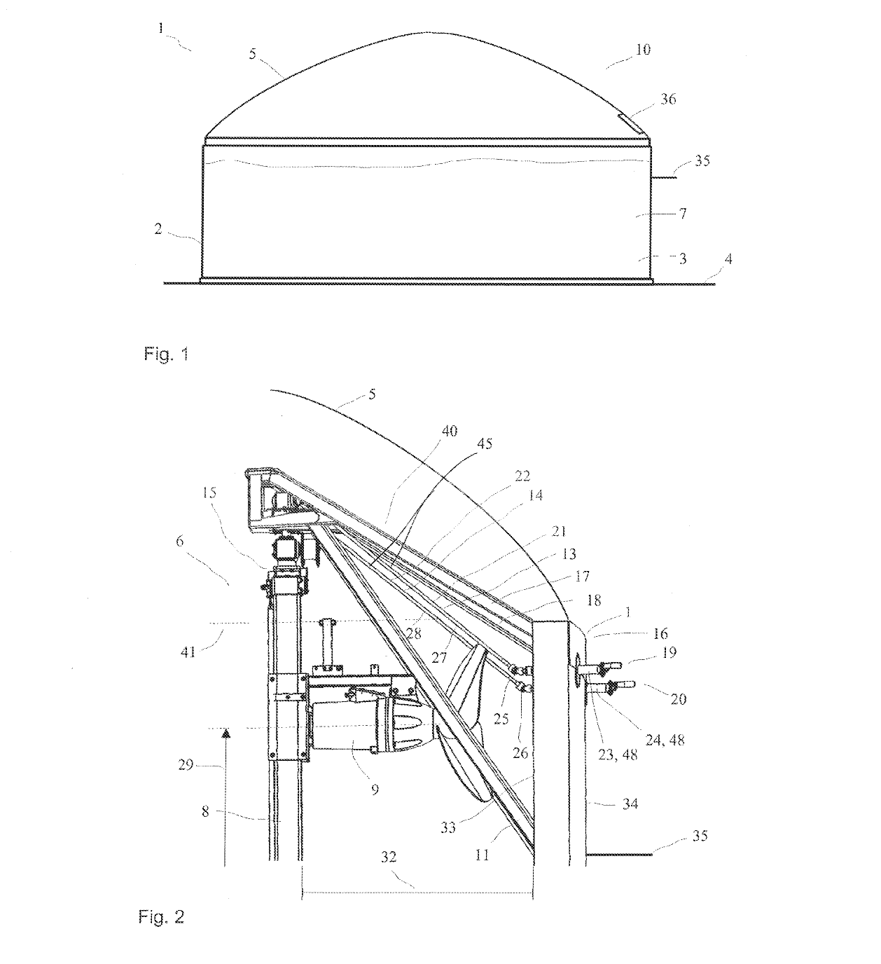

FIG. 2 a schematic cross section of the top end of the digester wall and an agitating unit pulled relatively far upwardly;

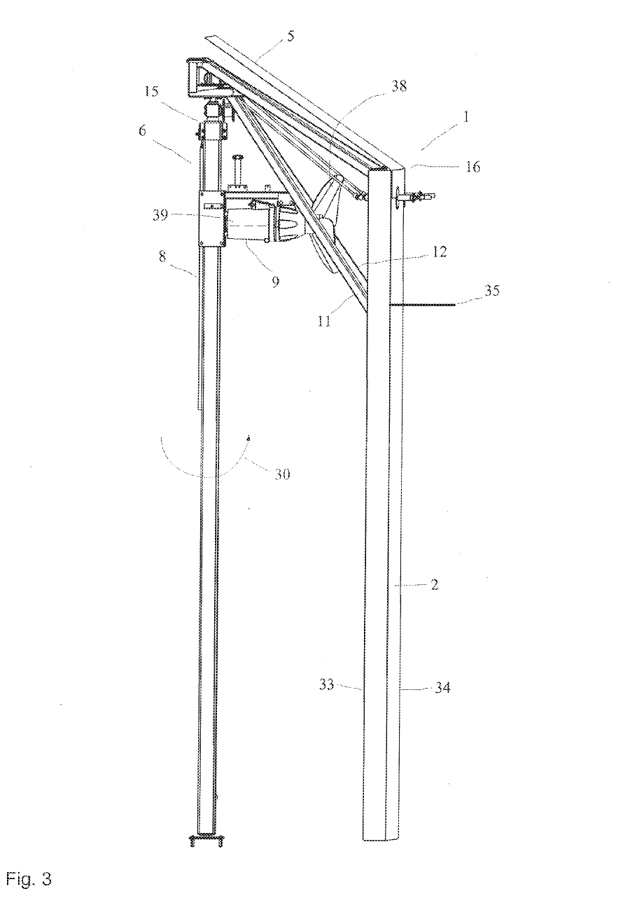

FIG. 3 a cross section of the entire digester wall height;

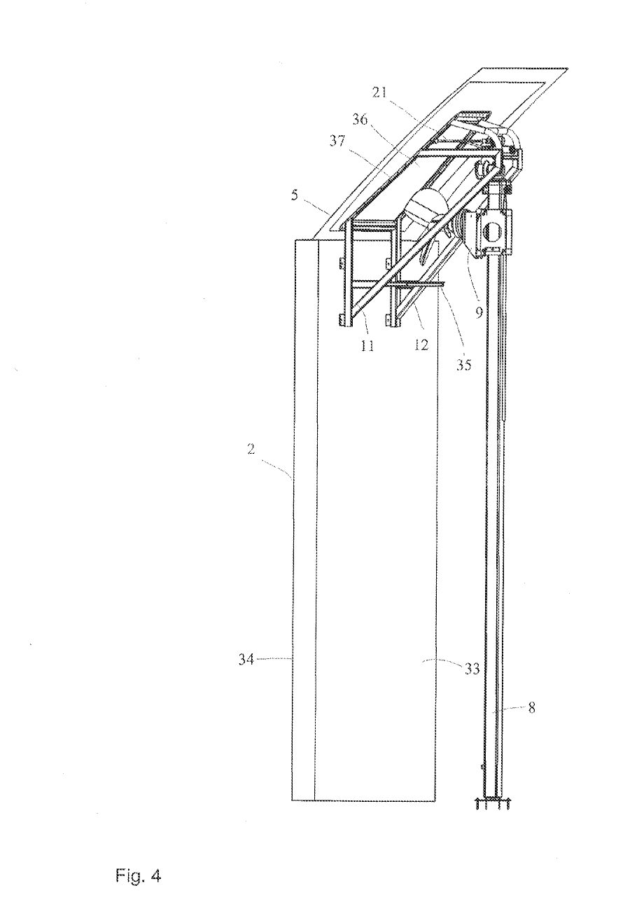

FIG. 4 a perspective inside view of another exemplary embodiment;

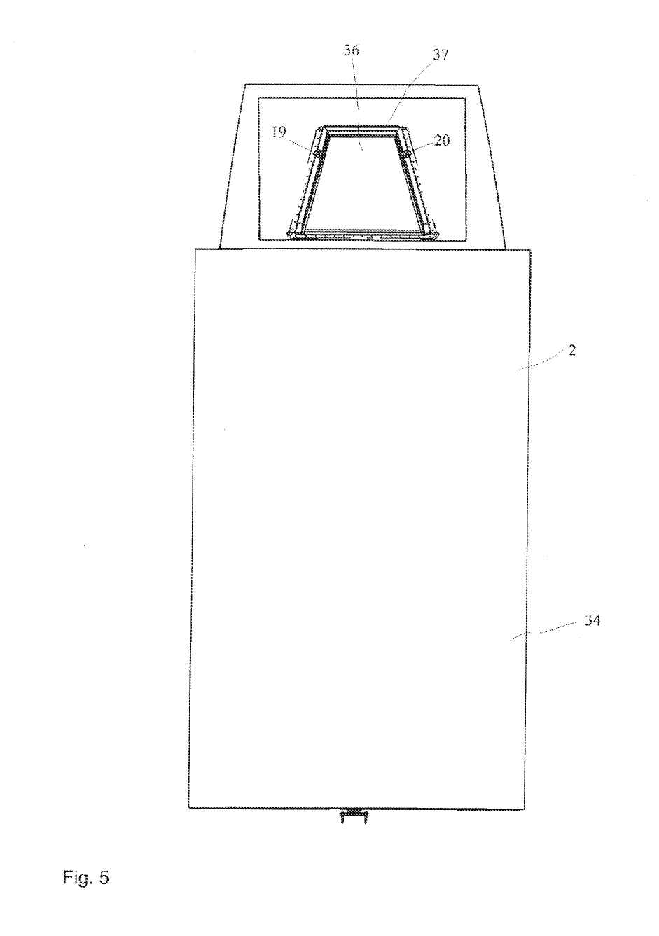

FIG. 5 an outside view of the digester wall segment in FIG. 4;

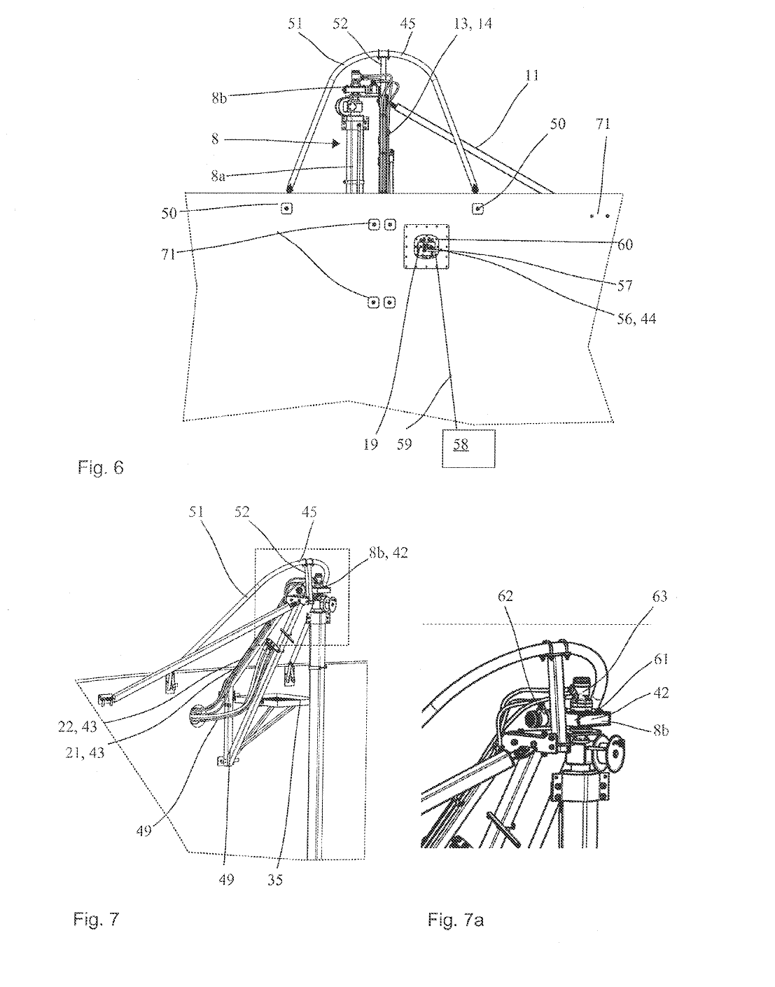

FIG. 6 a front view of another embodiment of a digester according to the invention in a purely schematic illustration;

FIG. 7 a perspective view of the digester according to FIG. 6 in a purely schematic illustration;

FIG. 7a an enlarged detail of the area marked in dotted lines in FIG. 7;

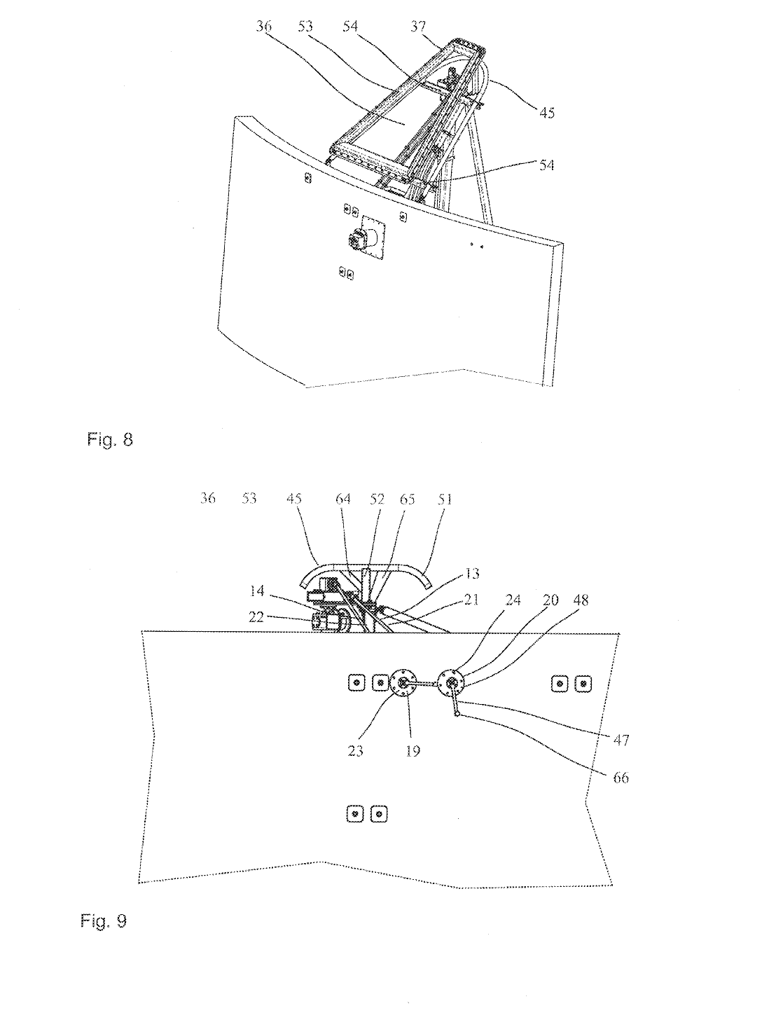

FIG. 8 a perspective inside view of the digester according to the invention according to FIGS. 6 and 7 in a purely schematic illustration;

FIG. 9 a front view of another embodiment of a digester according to the invention;

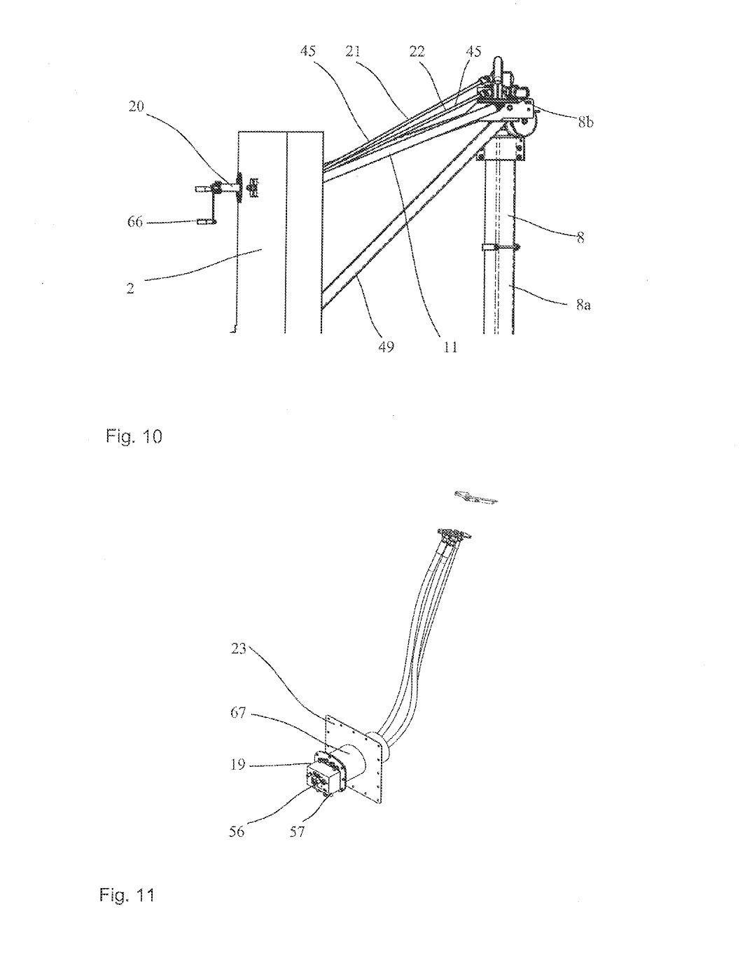

FIG. 10 a detail of the digester according to FIG. 9 in a schematic side view;

FIG. 11 a perspective view of a passage device in a purely schematic illustration;

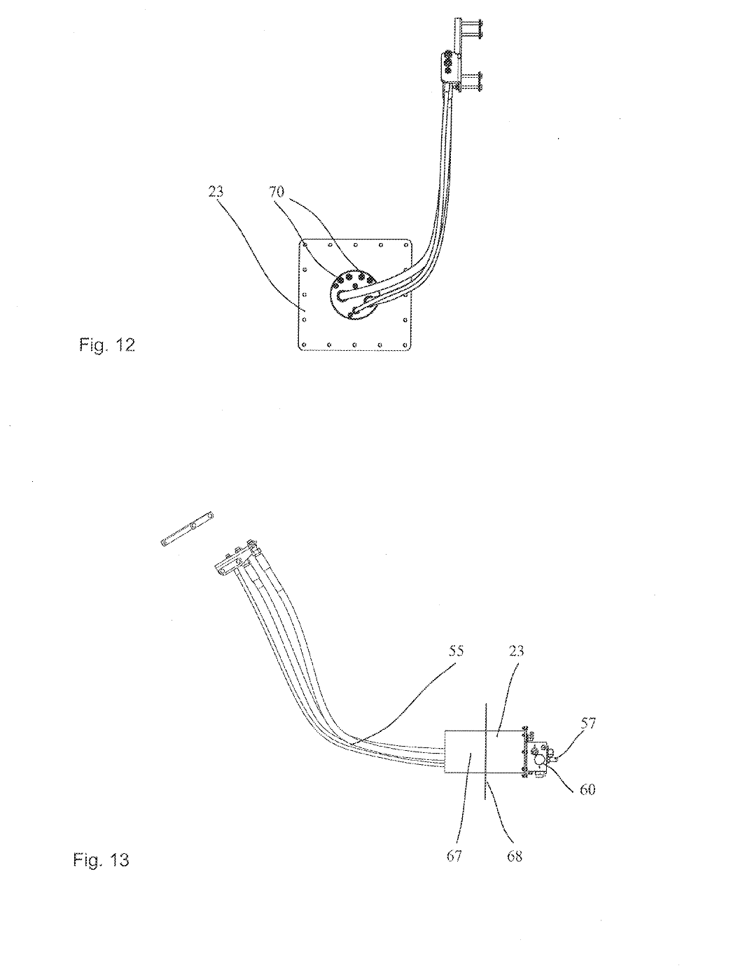

FIG. 12 the passage device according to FIG. 11 in a purely schematic rear view; and

FIG. 13 the passage device according to FIGS. 11 and 12 in a purely schematic side view.

Two exemplary embodiments will now be discussed with reference to the figures. FIG. 1 shows a simplistic side view of a digester 1 of a biogas plant 10. The digester 1 is preferably about circular in cross section and is presently provided with a circumferential digester wall 2. The digester roof 5 is formed in particular by a flexible material extending from the edge upwardly so as to obtain a domed, e.g. quarter-sphere or hemisphere or another curved structure of the tank roof 5. The inclination angle of the digester roof 5 depends on the specific conditions and may be 15° or more and in particular 30 or 45° or more. Preferably the digester roof 5 is at least partially and in particular entirely removable to render the digester interior 3 accessible.

In the digester interior 3 a substrate 7 is provided when in operation.

At least one servicing opening 36 may be provided in the digester roof 5 for example for servicing an agitator disposed in the digester interior 3. A platform 35 may be attached for example on the outside surface 34 or the inside surface 33 of the digester wall 2 for an operator to stand on.

FIG. 2 shows a partial cross section of the digester wall 2 showing the agitator device 6 with the agitating unit 9 provided thereat.

The agitating unit 9 is configured as a submersible motor agitator which may be driven electrically or for example hydraulically. In this exemplary embodiment the agitating unit 9 is provided with a plurality of e.g. three agitator blades 38 which rotate when the agitating unit 9 is in operation, thus causing a thorough mixing of the substrate 7 in the digester interior 3. This allows homogenization and enhanced efficiency.

The agitating unit 9 is retained height-adjustable on a guiding unit 8, the guiding unit 8 in the present exemplary embodiment comprising a supporting mast 8a and a transmission device 8b. This supporting mast 8a is provided by a hollow profile.

The agitating unit 9 can be conveyed upwardly from the position illustrated in FIG. 2 until the agitating unit 9 abuts the top end 15 of the guiding unit 8. The agitating unit 9 can be lowered downwardly along the guiding unit 8 until the agitator blades 38 are just a short distance above the digester floor.

The agitating unit 9 is provided pivotable together with the approximately rectangular supporting mast or the guiding unit that is approximately rectangular in cross section or shows any other shape. The guiding unit 8 can rotate about its longitudinal axis for pivoting the agitating unit 9 and for adjusting an agitating angle. This allows to align the agitating unit 9 in any desired direction.

Other than the guiding unit 8 the agitator device 6 also comprises support units 11 and 12 which in combination form a supporting frame with which to fasten and retain the agitator device 6 to the inside surface 33 of the digester wall 2. The support units 11 and 12 each comprise one or more supporting rods 17 and 18, in this case extending upwardly at an angle. This allows to effectively support the guiding unit 8 while at the same time allowing the agitating unit 9 to be displaced farther upwardly than has been the case in the agitator devices known thus far.

The guiding unit 8 or its top end 15 is disposed in the digester interior 3. This means that the guiding unit 8 does not penetrate the digester roof 5. The entire guiding unit 6 remains beneath the digester roof 5. This allows a considerably simpler structure of the digester roof 5 since its construction is not required to be designed for additional loads and stresses. A standard roof may be employed which considerably decreases costs. At the same time the construction according to the invention allows to displace the agitating unit 9 beyond the top end 16 of the digester wall 2 to bring it to the servicing position.

The distance 32 of the guiding unit 8 from the inside surface 33 of the digester wall 2 is dimensioned such that the agitating unit 9 can be pivoted toward the digester wall 2. The agitating unit 9 can be pivoted completely. Thus the agitator blades 38 when in the servicing position are disposed the closest possible to the digester wall 2 so as to allow ease of service.

The fact that when in the servicing position the agitator blades 38 extend beyond the top end 16 of the digester wall 2 at least in part allows a maintenance technician standing on the platform 35 to comparatively comfortably exchange the agitator blades 38.

The supporting frame consisting of the support units 11 and 12 with the supporting rods 17 and 18 is only attached to the digester wall 2. This exemplary embodiment does not provide for any connection with the digester roof 5.

Adjustment devices 13 and 14 are provided for adjusting a height 29 of the agitating unit 9 and for adjusting a rotational angle 30 of the agitating unit 9. The adjustment device 13 serves to adjust the height 29 of the agitating unit 9 and the adjustment device 14 serves to adjust the rotational angle 30 of the agitating unit 9. Basically it is also possible to provide the adjustment device 13 for adjusting the rotational angle and the adjustment device 14, for adjusting a height 29 of the agitating unit 9.

The height 29 of the pivot axis 39 of the agitating unit 9 may be adjusted up to the maximum height 41 where the housing of the agitating unit 9 abuts against the top end. In this case the maximum height 41 of the pivot axis 39 is higher than the top end 16 of the digester wall 2. Optionally the entire agitating unit 9 may be displaced to a height above the top end 16 of the digester wall 2.

The two adjustment devices 13 and 14 are substantially comparable in structure.

The adjustment device 13 comprises a control unit 19 on the outside surface 34 of the digester wall 2. The control unit 19 is followed by a gastight passage device 23 extending gastight through the digester wall 2. The passage device in the digester shown is configured as an articulated passage 48. On the inside surface 33 of the digester wall the gastight passage device 23 is followed by a cardan joint device acting as a deflecting device 25 which causes an angular deflection of the rotary motion. Starting from the cardan joint device acting as a deflecting device 25 the coupling unit 27 extends upwardly at an incline. The cardan joint device acting as a part of the deflecting device 25 together with the coupling unit 27 form an adjusting unit 21 which in this instance is mechanical, comprising a rod 45.

The adjustment device 14 comprises a control unit 20 disposed on the outside of the digester wall 2 and passing through the digester wall 2 by way of a gastight passage device.

The inside surface 33 of the digester wall 2 is provided with a cardan joint device acting as a deflecting device 26 that forms a part of an adjusting unit 22. The adjusting unit 22 is again mechanical, comprising a rod 45. The cardan joint device, being a component of the deflecting device 26, is connected with a coupling unit 28 extending upwardly at an incline.

The coupling units 27 and 28 are connected at their upper ends with transmission elements or transmission devices causing deflection of the coupling movements. This causes transmission of the rotational movement of the control unit 19 to height adjustment of the agitating unit 9 and of a rotational movement of the control unit 20 to a pivoting motion of the guiding unit 8 and thus of the agitating unit 9 as well.

The agitating unit 9 may be retained by a rope on the top end 15 of the guiding unit 8 where the rope is wound onto a spool. Rotation of the control unit 19 causes rotation of the spool.

FIG. 3 shows the entire height of the digester wall 2. The guiding unit 8 is accommodated at its lower end for rotation at the digester bottom to enable pivoting the agitating units 9. One can also see that the digester roof 5 extends above the agitator device 6 and is not connected therewith.

FIG. 4 shows another exemplary embodiment where one can see a perspective inside view of a section of the digester wall 2. Again, the agitating unit 9 is height-adjustably disposed on a guiding unit 8 that is disposed pivotable on the digester bottom. A supporting frame holds the guiding unit 8 at its top end. The supporting frame 40 comprises two support units 11 and 12 each comprising one or more supporting rods 17 and 18. Or else it is possible to provide one support unit only and no supporting frame. The support unit may for example have a disk-type configuration, pivotally guiding the guiding unit 8.

A platform 35 may be provided or positioned as needed on the support units 11 and 12, in the interior of the digester 1. The platform may optionally serve for an operator to stand on. Or else it is possible for the platform 35 to serve for placing items thereon during servicing.

Unlike the exemplary embodiment according to FIGS. 1 and 2 the agitator device 6 comprises a servicing frame 37 incorporated into the digester roof 5 and providing the servicing opening 36. The support units 11 and 12 carry the servicing frame 37.

In this configuration it is possible for the adjusting units 21 and 22 to not extend downwardly toward the digester wall 2 for height adjustment and for angular adjustment of the agitating unit 9 but to be aligned directly toward the servicing frame 37 where gastight passage devices 23 and 24 are provided with which to transmit the rotary motion for adjusting the agitating unit 9 from the exterior to the interior.

FIG. 5 shows an exterior view of the segment of the digester wall 2 according to FIG. 4 with the servicing frame 37 with the servicing opening 36 recognizable above the top end 16 of the digester wall 2. Moreover the control units 19 and 20 can be seen on the servicing frame 37. The components shown may be provided with a mechanical crank handle as required for adjusting the height and pivot position of the agitating unit 9.

In all the configurations the control units 19 and 20 may be provided with at least one display on which the rotational angle 30 or the height 29 of the agitating unit 9 can be seen.

On the whole the invention provides a digester 1 respectively an agitator device 6 to obtain a low-cost structure and simple operation. The invention dispenses with the need of incorporating the servicing opening 36 into the tank roof 5. It is also possible for the agitator device 6 to extend entirely beneath the digester roof 5. Nevertheless the maximum height of the agitating unit 9 can be adjusted so that the agitating unit 9 extends upwardly beyond the top end 16 of the digester wall 2. This simplifies maintenance of the agitating unit 9, in particular if a servicing opening is provided in the digester roof 5 above the agitator device 6. Specific configurations allow to provide the servicing opening 36 in a servicing frame 37 that is connected with the agitator device 6.

It is another advantage of the construction according to the invention that no ropes pass through the tank wall. For maintenance the agitator may be rotated so as to be vertical along the tank wall. Maintenance does not require to lower the filling level. In operation the tank wall 2 may virtually be filled up to the maximum level. It is possible to provide a position indicator for the agitating unit 9. The maximum fill height possible allows to increase the utilization factor of the tank so as to virtually reduce the construction costs.

The pivoting range of the agitating units 9 may be 180°, 270°, 300°, 330° or 360° or more. For maintenance the agitating unit 9 may be rotated so as to be vertical along the tank wall. Optionally the agitator device 6 may be incorporated into the digester roof 5.

The construction according to the invention is moreover more low-maintenance since the gastight passage devices are permanently tight and do not require lubrication on a regular basis. Also it is impossible for a water cup to dry out.

Virtually all the mechanical components are disposed in the digester tank. Optionally at least one viewing window may be provided.

The FIGS. 6 to 8 schematically show another exemplary embodiment of a digester 1 according to the invention. The basic structure of the digester 1 shown is similar to the digester 1 described above.

The digester 1 has a guiding unit 8 disposed therein comprising a supporting mast 8a and a transmission device 8b with the transmission device 8b configured as a hydraulic transmission 42. The guiding unit 8 is accommodated and held in position on the digester wall 2 by means of the console 49. The console 49 and the guiding device 8 are supported by means of a support unit 11 acting as a lateral support strut. A platform 35 is also attached to the console 49 for a technician to stand on during maintenance.

As can be readily seen in the FIGS. 6 and 7, a supporting bar device 45 is provided above the guiding device 8 that is firmly connected with the digester wall 2 by way of two attachment ties 50. The safety bar device in the shape of a safety bar 51 is connected and supported on the guiding unit 8 by means of a support 52.

The safety bar device 45 is to prevent damage to the digester roof 5 when the film roof 5 collapses as the system is shut down or in case of failure.

Then the film roof 5 will fall onto the safety bar 51 and cannot be damaged or even perforated by the guiding unit 8 or any of its components.

In the digester wall one can identify more attachment ties by means of which the console 49 and the support unit 11 are fastened to the digester wall 2.

FIG. 8 further shows that the safety bar device 45 accommodates a servicing frame 37 to provide a servicing opening 36. In the exemplary embodiment shown the servicing frame 37 is a clamping frame 53 consisting of a pair of frame components between which the various layers of the film roof 5 are clamped. This allows, among other things, to subsequently insert a servicing opening 36 into an existing film roof 5 using comparatively simple means.

This exemplary embodiment provides for two cross beams 54 to firmly accommodate the clamping frame 53 which are connected with the safety bar device 45 and which securely support the clamping frame 53. The clamping frame 53 can be e.g. screw-fastened to one or both of the cross beams. Other configurations may provide for the clamping frame 53 to be provided at or attached to the digester 1 in some other way.

In FIGS. 6 to 8 the agitating unit 9 is hydraulically displaced and pivoted. To this end the gear transmission 8b is configured as a hydraulic transmission 42 that is operated by means of hydraulic adjustment devices 13, 14.

This hydraulic transmission 42 is a twin-top transmission 61 having two transmission modules 62, 63, one of which being in charge of height adjustment and the other, of pivoting the agitating unit 9. The transmission modules 62, 63 can be identified in the enlarged illustration in FIG. 7a.

The adjustment devices 13, 14 comprise adjusting units 21, 22 in the form of hydraulic hoses 43, an energy supply 55 for the agitating unit 9, and a shared control unit 19.

The control unit 19 comprises a hydraulic pump 44 with which to raise and lower and pivot the agitating unit 9.

To this end the control unit 19 is provided with a manual pump 56 having a lever 57. The lever 57 serves to manually operate the adjustment devices 13, 14. The lever 57 can be used for pumping wherein the lever 57 can be folded down in two positions for adjusting the height of the agitating unit 9 or for pivoting the agitating unit 9.

In all the configurations the height and/or the pivoting angle of the agitating unit(s) 9 can be displayed on a position indicator (not shown).

Since in the exemplary embodiment shown the control unit 19 is disposed high up on the digester 1 in the explosion-prone region, using motorized pumps is critical.

Therefore a motorized pump 58 may be connected with the control unit 19 via an interface 60 that is displaced by means of a hydraulic line 59 out of the danger zone e.g. onto the floor or into a service room. Then e.g. a mobile motor or a mobile hydraulic pump may be connected on the floor adjacent to the digester 1.

FIGS. 9 and 10 schematically illustrate another exemplary embodiment of a digester 1 according to the invention. The basic structure of the digester 1 and the agitator device 6 substantially correspond to the exemplary embodiment according to the FIGS. 1-3.

The guiding unit 8 accommodating the agitating unit 9 is received and held in position in the digester wall 2 via a console 49. A support unit 11 provides for lateral support.

The digester 1 according to FIGS. 9 and 10 provides for the agitating unit 9 to be mechanically varied in height and pivoted. Two adjustment devices 13, 14 are provided, each comprising an adjusting unit 21, 22 in the shape of a rod 45 and a control unit 19, 20 each.

The operating devices 19, 20 comprise a crank device 47 each, which in the exemplary embodiment shown are manual cranks 66.

The control unit 19 allows to adjust the height of the agitating unit 9 on the supporting mast 8a of the guiding unit 8. The control unit 20 allows to adjust the height of, and to pivot, the agitating unit 9.

The two passage devices 23, 24 are designed as articulated passages 48 which are also provided with deflecting devices 26 and coupling units 27, 28 not shown in detail.

In this exemplary embodiment a safety bar device 45 is also provided at least in sections above the guiding device 8 for protecting the film roof 5 from any damage.

As the FIGS. 9 and 10 clearly show, the guiding device 8 protrudes above the digester wall 2 to an insubstantial extent only. Moreover, no servicing opening 36 is provided which would e.g. require a clamping frame 53.

Therefore a very small safety bar 51 is sufficient in this exemplary embodiment, fastened to the guiding device 8 only via the supports 52, 64, 65. In the digester shown the safety bar 51 does not need to be affixed to the digester wall 2.

The specific configuration having a guiding device 8 in a very flat construction and the rudimentary safety bar 51 allow to keep acquisition costs down.

A passage device 23 according to the invention for a digester 1 with an agitating unit 9 that can be hydraulically adjusted in height and pivoted is schematically illustrated in the FIGS. 11 to 13.

The passage device 23 comprises a tube section 67 that serves as a wall duct 68 through the digester wall 2. An attachment plate 69 is provided on the tube section 27 for fixating the passage device 23 by means of which the passage device 23 is e.g. screwed onto the digester wall 2.

In the exemplary embodiment shown the passage device 23 also comprises the control unit 19 including a hydraulic manual pump 56 with a lever 57 and an interface 60 for connecting a hydraulic line 59.

The hoses illustrated in the figures provide for the energy supply 55 for the agitating unit 9. The hydraulic hoses 43 which serve for adjusting units 21, 22 of the adjustment devices 13, 14 for actuating the hydraulic transmission 42 are not shown in detail in the figures.

In FIG. 12, which shows a rear view of the passage device 23, one can identify the connectors 70 for the hydraulic hoses 43 required for adjusting and pivoting.

On the whole the agitator devices 6 according to the invention can be operated mechanically, hydraulically, and/or pneumatically. This allows to compose a suitable agitator device 6 depending on the digester 1 and the budget. The agitator devices 6 according to the invention also allow to combine various operating methods.

List of reference numerals:

| | |
|---|---|
| 1 | digester |
| 2 | digester wall |
| 3 | digester interior |
| 4 | horizontal |
| 5 | digester roof |
| 6 | agitator device |
| 7 | substrate |
| 8 | guiding unit |
| 8a | supporting mast |
| 8b | transmission device |
| 9 | agitating unit |
| 10 | biogas plant |
| 11 | support unit |
| 12 | support unit |
| 13 | adjustment device |
| 14 | adjustment device |
| 15 | top end of 8 |
| 16 | top end of 2 |
| 17 | supporting rod |
| 18 | supporting rod |
| 19 | control unit |
| 20 | control unit |
| 21 | adjusting unit |
| 22 | adjusting unit |
| 23 | passage device |
| 24 | passage device |
| 25 | deflecting device |
| 26 | deflecting device |
| 27 | coupling unit |
| 28 | coupling unit |
| 29 | height |
| 30 | rotational angle |
| 31 | angle adjustment device |
| 32 | distance |
| 33 | inside surface |
| 34 | outside surface |
| 35 | platform |
| 36 | servicing opening |
| 37 | servicing frame |
| 38 | agitator blade |
| 39 | rotation axis of 8 |
| 40 | supporting frame |
| 41 | maximum height |
| 42 | hydraulic transmission |
| 43 | hydraulic hose |
| 44 | hydraulic pump |
| 45 | safety bar device |
| 45 | rod |
| 47 | crank device |
| 48 | articulated passage |
| 49 | console |
| 50 | attachment tie |
| 51 | safety bar |
| 52 | support |
| 53 | clamping frame |
| 54 | cross beam |
| 55 | energy supply |
| 56 | manual pump |
| 57 | lever |
| 58 | motorized pump |
| 59 | line |
| 60 | interface |
| 61 | twin-top transmission |
| 62 | transmission module |
| 63 | transmission module |
| 64 | support |
| 65 | support |
| 66 | manual crank |
| 67 | tube section |
| 68 | wall duct |
| 69 | attachment plate |
| 70 | connecting device |
| 71 | attachment tie |

1. Digester (1) of a biogas plant (10) having at least one digester wall (2) and at least one digester interior (3) and at least one digester roof (5) inclined to the horizontal (4) and configured in particular flexible and having at least one agitator device (6) for stirring a substrate (7) in the digester interior (3), the agitator device (6) comprising at least one guiding unit (8), at least one support unit (11, 12), and at least one adjustable agitating unit (9) accommodated on the guiding unit (8), the support unit (11, 12) being attached to the digester wall (2) for supporting the guiding unit (8), wherein the agitating unit (9) is provided height-adjustable, and wherein at least one adjustment device (13, 14) is provided for adjusting the agitating unit (9), characterized in that the top end (15) of the guiding unit (8) extends up to and beyond a top end (16) of the digester wall (2) and terminates beneath the digester roof (5) inside the digester interior (3).

2. The digester according to claim 1 wherein at least a part of the agitating unit (9) can be displaced upwardly beyond the top end (16) of the digester wall (2).

3. The digester according to claim 1 or 2 wherein the rotation axis (17) of the agitating unit (9) can be displaced upwardly beyond the top end (16) of the digester wall (2).

4. The digester according to claim 1 wherein the agitating unit (9) can be displaced upwardly entirely above the top end (16) of the digester wall (2).

5. The digester according to claim 1 wherein the support unit (11, 12) comprises at least one supporting rod (17, 18) extending upwardly from the digester wall (2) at an incline.

6. The digester according to claim 1 wherein the adjustment device (13, 14) comprises at least one control unit (19, 20) disposed external of the digester interior (3) and at least one adjusting unit (21, 22) disposed internal of the digester interior (3) which are coupled by means of at least one gastight passage device (23, 24).

7. The digester according to claim 1 wherein the guiding unit (8) comprises at least one supporting mast (8a) and at least one transmission device (8b).

8. The digester according to claim 1 wherein the adjusting unit (21, 22) is in functional connection with the transmission device (8b).

9. The digester according to claim 6 wherein the adjusting unit (21, 22) is mechanical, hydraulic, and/or pneumatic in design.

10. The digester according to claim 1 wherein the transmission device (8b) is a hydraulic transmission (42) and the adjusting unit (21, 22) comprises, or is formed as, at least one hydraulic hose (43) wherein the hydraulic hose (43) connects the hydraulic transmission (42) with the control unit (19, 20) which comprises at least one hydraulic pump (44).

11. The digester according to claim 1 wherein the adjustment device (13, 14) is mechanical, wherein the adjusting units (21, 22) are rods (45), and the control unit (19, 20) comprises at least one crank device.

12. The digester according to claim 11 wherein the gastight passage device (23, 24) is disposed beneath the digester roof (5) and in particular in the digester wall (2).

13. The digester according to claim 1 wherein the control unit (19, 20) is disposed beneath the top end (15) of the guiding unit (8) and in particular also beneath the top end (16) of the digester wall (2).

14. The digester according to claim 1 wherein the adjusting unit (21, 22) comprises at least one deflecting device which deflecting device in particular comprises, and/or is configured as, at least one cardan joint device (25, 26) and/or at least one angular drive device and/or at least one flexible shaft.

15. The digester according to claim 1 wherein the adjusting unit (21, 22) comprises a coupling unit (27, 28) inclined to the horizontal (4).

16. The digester according to claim 14 wherein the deflecting device (25, 26) is disposed between the coupling unit (27, 28) and the passage device (23, 24).

17. The digester according to claim 15 wherein the agitating unit (9) is provided pivotable and wherein the pivoting angle (30) is adjustable by means of an angle adjustment device (31).

18. The digester according to claim 1 wherein a distance (32) of the guiding unit (8) from the inside surface (33) of the digester wall (2) is dimensioned such that the agitating unit (9) can be pivoted toward the digester wall (2).

19. The digester according to claim 1 wherein at least one platform (35) is provided at and in particular fastened to the digester wall (2) and/or the support unit (11, 12).

20. The digester according to claim 1 wherein at least one servicing opening (36) that locks gastight is provided in the digester roof (5).

21. The digester according to claim 1 wherein the servicing opening (36) is provided above the guiding unit (8).

22. The digester according to claim 1 wherein the servicing opening (36) is provided in a servicing frame (37) and wherein in particular the servicing frame (37) is provided with at least one gastight passage device (23, 24).

23. The digester according to claim 1 wherein at least one safety bar device (45) is provided beneath the digester roof.

24. The digester according to claim 22 wherein the servicing frame (37) is accommodated on the safety bar device (45).

25. The digester according to claim 1 wherein the adjustment device (13, 14) can be operated manually and/or motorized.

26. Agitator device (6) in particular for a digester of a biogas plant (10) for stirring a substrate (7) in the digester interior (3), comprising at least one guiding unit (8) and at least one support unit (11, 12), wherein the guiding unit (8) accommodates an adjustable agitating unit (9) and wherein the support unit (11, 12) is attached to a digester wall (2) for supporting the guiding unit (8), wherein at least one adjustment device (13, 14) is provided for displacing the agitating unit (9), characterized in that the adjustment device (13, 14) comprises at least one adjusting unit (21, 22) that is connected with at least one control unit (19, 20) via at least one gastight passage device (23, 24).

27. The agitator device (6) according to claim 26 wherein an adjusting unit (21) is provided for adjusting a height (29) of the agitating unit (9) and/or wherein an adjusting unit (22) is provided for adjusting an angle (30) of the agitating unit (9).

28. The agitator device (6) according to claim 26 wherein the adjustment device (13, 14) comprises at least one deflecting device (25, 26).

29. The agitator device (6) according to claim 26 wherein at least one adjusting unit (21, 22) is mechanical, hydraulic, or pneumatic in design.
